(19) United States
(12) Patent Application Publication
(10) Pub. No.: US 2002/0026656 A1
(43) Pub. Date: Feb. 28, 2002

(54) NOVEL PLANT TEST PROCEDURE TO DETECT NATURAL, SEMI-SYNTHETIC, SYNTHETIC COMPOUNDS AND PHYSICAL STRESS FACTORS THROUGH EXPRESSION OF DISTINCT RESPONSES

(76) Inventors: SUSHIL KUMAR, LUCKNOW (IN); SUMAN PREET SINGH KHANUJA, LUCKNOW (IN); MAHENDRA PANDURANG DAROKAR, LUCKNOW (IN); TIRUPPADIRIPULIYUR RANGANTHAN KUMAR, LUCKNOW (IN); ANITA GANGWAR, LUCKNOW (IN); AJIT KUMAR SHASANY, LUCKNOW (IN); SRILEKHA MISRA, KANPUR (IN); SHALINI MATHUR, MEERUT (IN); DHARMENDRA SAIKIA, LUCKNOW (IN)

Correspondence Address: JAMES J MURPHY, WINSTEAD SECHREST & MINICK, PC, 5400 RENAISSANCE TOWER, 1201 ELM STREET, DALLAS, TX, 75270, US

(21) Appl. No.: 09/246,862

(22) Filed: Feb. 8, 1999

(30) Foreign Application Priority Data
Dec. 9, 1998 (IN) 3704/DEL/98

(51) Int. Cl. C12N 15/82

(52) U.S. Cl. 800/278

(57) ABSTRACT

The present invention relates to a method for testing abiotic stress factors and bioactivity of natural, synthetic and semisynthetic compounds using Bacopa monnieri plant, which comprises growing the said plant and plant parts aseptically in MS 0 basal medium with agar in microcentrifuge tubes by adding the compounds to be tested either to the culture media or by spraying the said compounds on the plant or plant parts or on the said medium to detect the distinct morphological and cytological responses.

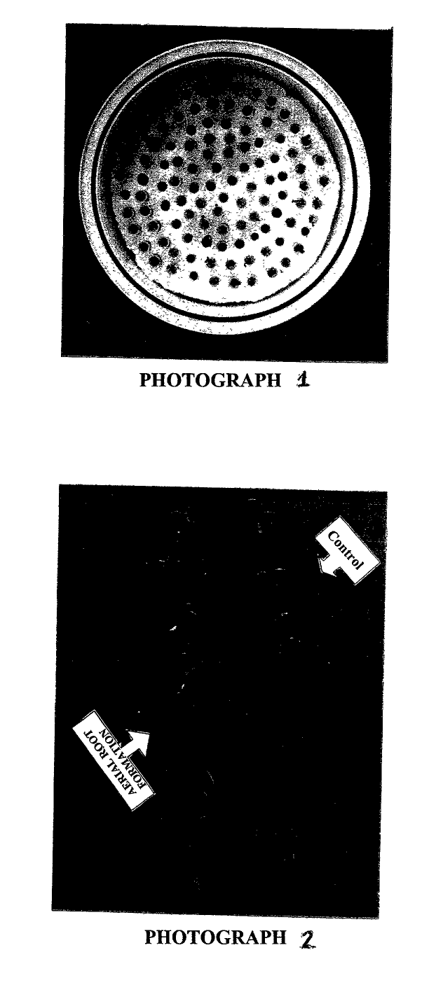

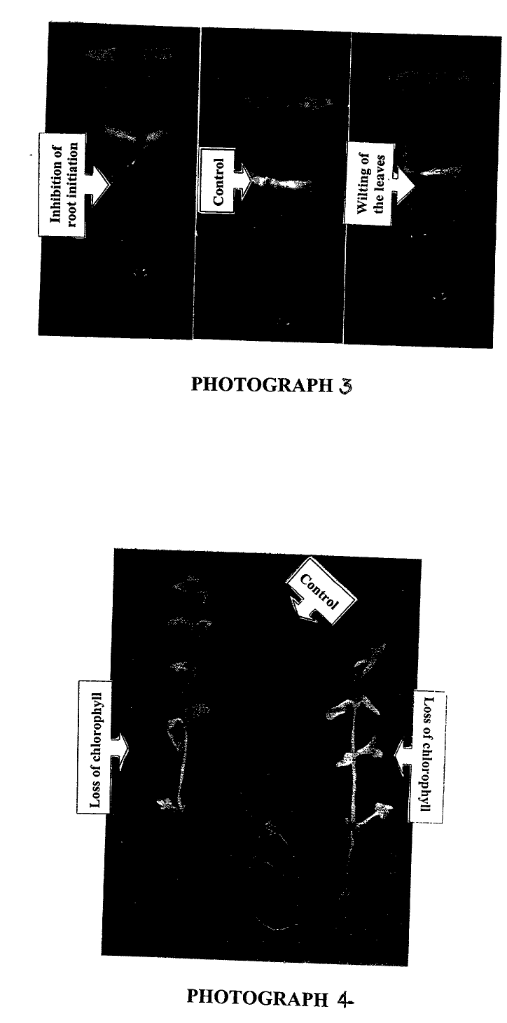

This application claims priority to application Ser. No. ______, filed with the Indian Patent Office on Dec. 9, 1998.

### FIELD OF THE INVENTION

The present invention relates to a method for testing bioactivity of the natural, synthetic, semisynthetic compounds and monitoring exposure to abiotic stress factors using Bacopa monnieri plant. More particularly, the invention relates to a method for novel plant test expression system using a rapid propagating Bacopa monnieri plant capable of detecting natural or synthetic compounds useful in promoting or inhibiting plant growth and other activities like, cell division, cytotoxic, weedicidal and anticancerous activities and also as a biosensor for monitoring the physical stress factors and hazards in the environment including ionising, and non ionising radiation (radioactivity), heat and cold shocks.

### BACKGROUND

Bioactivity testing for the chemical compounds whether natural, semisynthetic or synthetic and monitoring environment for the presence of physical stress factors and hazards like radioactivity, radiation and temperature shocks have been a major concern for all those who are concerned about the environment for the following main reasons:

Monitoring the compounds for their toxicity or pollution hazards before they are permitted to be released into any practical use in the environment.

Bioevaluating the compounds for their potentials in developing drugs or agrochemicals.

Biosensing the presence of physical extremities in the environment which could amount to physical damage/shock to the organisms living there.

The emphasis in the present day scenario is centered on natural compounds not only for their direct use but also for providing the biorational leads in developing semisynthetic compounds with higher efficacy. These biorational leads are expected to be more biocompatible and therefore, the appropriate tests have to be applied for both positive and negative effects. About 51 tests are well known to be approved for the use within OECD countries (Organization for Economic Co-operation and Development: Guidelines for Testing of Chemicals. OECD, Paris, 1981, for testing of chemical compounds. Some of the higher plant species have also been used for toxicity assessment of industrial wastewater including common duckweed (Lemna minor), lettuce, rice (Oryza sativa) and wheat etc. Wang W, 1990, Res. J. Water Pollut. Con. Fed. 62: 853-860; Wang W, 1991, Plants for Toxicity Assessment, second volume, J W Gorsuch, W R Lower, W Wang and M A Lewis, Eds. ASTM STP 1115, American Society for Testing and Materials, Philadelphia, pp 68-70). Single test has never been advocated as sufficient enough to be employed as biotesting procedure particularly, when the compound under question is to be evaluated for environmental toxicity/hazard (Fiskesjo G, 1982, Ph.D. Thesis, Institute of Genetics, University of Lund, Sweden).

In addition, use of a battery of tests rather than any single test in isolation has also been preferred to reduce the possibility of false negative and false positive results (de Serres, 1976, Mutation Research, 38: 165-176 and 355-358) . In fact, this opinion has always found the general consensus among the regulatory agencies as well (Committee 17,1975, Science, 187:503-514). Availability of several tests has resulted into their broad classification among four main classes (Maugh, 1978 Science, 201:1200-1205). These include:

The tests with micro-organisms.

The tests with intact organisms.

The tests with cultured/mammalian cells.

The tests for in-vitro activities.

In the above categories, for the tests with intact organism a very popular root tip assay system using Alliuin ceua sprouted bulbs was introduced by Levan 1938, while investigating the effects of colchioine. The usefulness of plant system in tests of chemicals even those which need metabolic activation has been emphasised (Vig B K, 1978, Environment Health Perspect. 27: 27-36) for the preliminary screening of new chemicals being introduced in to the environment. The Allium test has also been listed as a short term procedure for the detection of chemical carcinogens (Stich H F, 1975, Can. J. Genet. Cytol. 17:471-492). The positive results in these tests have been suggested to be considered as a warning that the tested chemicals may be a risk to human health. The first attempt to approach the problem of genotoxical effect of environmental chemicals was on a commercial fungicide (called Be Toxin) containing mercury compounds (Levan A, 1945, Nature, 156:751). Subsequent studies on effect of mercury halogenides in Allium test have substantiated the confirmation with compounds like methyl mercury chloride (MMC) and ethyl mercury chloride (EMC) etc. (Levan A, 1951, C.S.H. Symp. Quant. Biol. 16:233-243; Fahmy F Y, 1951, Ph.D. thesis, Inst. Genet., Lund, Sweden; and Remel, C. 1969, Hereditas 61:208-230). The original form of test wherein outgrowth of root tips from onion bulbs in fresh water was followed by treatment with test chemical solution has undergone several modifications to have several replicates together (Fiskesjo G, 1975, Vatten 31(4):304-316 and Fiskesjo G, 1981, Vatten 37(3):232-240).

The system although very useful to study the responses of a variety of cell division inhibiting substances, has certain inbuilt limitations which are as follows:

The parameters which can be studied using this test procedure are mainly confined to cytological observations including cell division.

This test is not usable for assessing the growth promotion activities since the responses are observed only in the root tips and thus do not take in to consideration the effects on aerial parts of the plant as a direct measure.

It requires independent onion bulbs in the sprouting stage for each treatment of every replication which amount to large bulk of starting material and becomes impractical when thousands of samples are to be tested.

The root sprouting of bulbs is not possible throughout the year under ambient condition and therefore, the experiment would require storing the right stage of bulb under controlled conditions.

Each bulb has to be sprouted on top of a tube/flask filled with buffer having minimum 15×20 mm diameter and therefore requires larger volumes of buffer or medium for testing the compounds.

### Objects of the Invention

The object of the invention is to use the rapid growing herb Bacopa monnieri in the bioassay procedures for detecting distinct plant response in presence of test compounds and extracts in a convenient, rapid and repetitive manner. The experiments were specifically designed to analyze the use of procedure based on the observations and interpretations of the Bacopa test which is unique and efficient for its multi-purpose applications providing suitable alternative for not only traditional root tip assays (Alliurn test) and shoot growth assays (coleoptile procedure) but also having its applicability in assessing chemical compounds with cytotoxic or weedicidal effects. Another object of the invention is to provide an unique process to monitor various elements of environmental pollution.

### Detailed Description of the Invention

The present invention provides a novel test process by utilising a fast growing plant Bacopa monnieri. The effects of natural, semisynthetic and synthetic compounds and physical stress factors on the living system can be tested in a rapid and much simpler way. The chemical compounds whose effects can be tested by this process are growth promoters, growth inhibitors, cytotoxic and weedicidal compounds, cell division inhibitors and antimitotic/anticancerous compounds. The effects of physical factors like temperature, ultra violet and gamma irradiation on living systems can be tested using this process. The novel process is important as it can be used as a biosensor in the present world of environmental pollutants and biohazards.

The invention of this test procedure is a consequence of planned experiments carried out for developing a simple, repeatable, and rapid procedure using plants or plant parts to detect the biological activities of various natural and synthetic compounds.

The Applicants initiated the experiment to search a better plant system which is available in plenty, grows rapidly, propagates vegetatively and can show rooting response with aerial parts to test the effect of pollutants on living systems. One such plant Bacopa monnieri (family: Scrophulariacea) which is used at CIMAP in experiments for medicinal values was found to be responding very fast for all type of explants under, culture conditions. This plant is small prostrate herb with ascending branching and grows wildly throughout the plains of India including coastal region. Therefore from the point of view of availability of plant material as well as its quick response under controlled conditions, it was tested for specific responses to develop a procedure of biotesting the natural and synthetic compounds as well as biosensing the environment for physical stress factors. While developing the plant test system the drawbacks of the other system were kept in mind. As mentioned earlier, the Applicants used the rapid growing herb Bacopa monnieri in the bioassay procedures for distinct responses in presence of test compounds and extracts in a convenient, rapid and repeatable way the experiments were specifically carried out to analyze the use of procedure based on the observations and interpretations about the Bacopa test being unique and efficient for its multi-purpose applications providing better alternative to not only the traditional root tip assays (Allium tested) and shoot growth assays (coleoptile procedure) but also its applicability in assessing chemical compounds with cytotoxic or weedicidal effects.

Initially the experiments were designed to observe the response of leaf, internode and cuttings against growth promoters (auxins: 2,4-D & Indole acetic acid) and growth inhibitors (cytokinins: 6-Benzyl amino purine & kinetin). The basal medium for studying the response was MS 0 (Murashige T and Skoog F, 1962, Physiol. Planta. 15: 473-497) supplemented with these growth regulators at the rates of 0.5 and 1.0 mg/liter along with a control of just the basal medium. In case of leaf explant as well as internodal parts profuse rooting was observed for Indole acetic acid, while in case of 2,4-D small thick roots got initiated at the internodes. For BAP, green callus developed in all three explants followed by shoot regeneration. In control experiment, root initiation occurred from leaves and cuttings but was conspicuously delayed by 4-6 days.

Another set of experiments was conducted to ascertain whether the initiated roots or even the root initiation process itself could be used as a parameter for cell division inhibition studies to compare its use as a system to replace the Allium test (Fiskesjo G. 1975, Vatten 31(4):304-316). Since plant growth in a single pot can yield thousands of twig cuttings and each cutting could be used as the starting material for root initiation as well as the root tip assays it becomes an advantage for large scale rapid monitoring of the cell division inhibition. In these experiments, various concentrations of taxol ranging from 1 to 100 μg/ml were taken. The roots were initiated from large number of cuttings in the basal medium by culturing for one week. For root tip assays the twigs with initiated roots were dipped in basal medium broth containing different levels of taxol and related compounds for different time intervals followed by fixation, staining in heamatoxylin for two hours and mounting on slides followed by observation for mitotically dividing cells. For root initiation assays, on the other hand, the fresh twigs were directly planted in to 1.5 ml microcentrifuge tube containing basal medium plus taxol at various concentrations. In case of root initiation assays, it was observed that the twigs did not initiate the roots at all even after 14 days and there was no shoot growth as well. Rather the wilting and later withering was observed in treated samples. In case of a root tip assay with taxol, a substantial reduction in the mitotic index was observed for taxol at the rate of 10 μg/ml. The observed mitotic index showed 2.55 fold reduction compared to untreated control sample. Taxol at the rate of 100 μg/ml inhibited almost complete cell division in the root tips.

For studying the response of Bacopa twigs as biosensor system against physical factors, tests were conducted to see the effect of temperature shock and radiation exposure. It was observed that temperatures between 0 to 4° C. caused early root initiation while temperature shock of −20° C. beyond 10 min caused the death of plantlets. In case of higher temperature shocks the plants died upon a shock of more than 1 min at 50° C. or more than 20 seconds at 80° C. In UV exposure experiment the twigs could tolerate UV radiation up to 2 hrs but beyond this time the survival was drastically affected and after 8 hrs 100% mortality was observed.

Accordingly, the invention provides a novel process wherein a method for testing abiotic stress factors and bioactivity of the natural, synthetic and semisynthetic compounds Bacopa monnieri plant, which comprises growing the said plant and plant parts aseptically in MS basal medium with agar in microcentrifuge tubes by adding the compounds to be tested either to the culture media or by spraying the said compounds on the plant or plant parts or on the said medium to detect the distinct morphological and cytological responses.

In one embodiment, the compounds to be tested are used either in pure or crude extract form.

In another embodiment, the morphological responses to be detected are selected from the group consisting of callus initiation, shoot induction, root induction, necrosis and death.

In yet another embodiment, the cytological responses to be detected are selected from the group consisting of mitotic index and C-mitosis.

In yet another embodiment, the compounds used for bio-testing includes growth promoters selected from the group consisting of auxins and cytokinins.

In yet another embodiment, the auxins used are selected from the group consisting of but not limited to Indole-3-acetic acid 2, 4-Dichlorophenoxy acetic acid.

In yet another embodiment, the cytokinins used are selected from the group consisting of but not limited to 6-Benzyl amino purine and kinetin.

In another embodiment, the compounds used for bio-testing comprises growth inhibitors selected from the group consisting of nalidixic acid and abscisic acid.

In another embodiment, the compounds used for bio-testing include weedicidal compounds.

In yet another embodiment, the weedicidal compound used is but not limited to 2,4-Dichlorophenoxy acetic acid.

In other embodiment, the compounds used for bio-testing include antimitotic and anticancerous compounds.

In another embodiment, the antimitotic and anticancerous compounds are selected from the group consisting of but not limited to taxol, trichothecene, vincristine, vinblastine and nalidixic acid.

In further embodiment, the compounds used for bio-testing include cytotoxic compounds.

In yet another embodiment, the cytotoxic compounds are selected from the group consisting of but not limited to menthol, mint oil, artemisia oil, basil oil and essential oil components.

In yet another embodiment, the abiotic stress factors used are selected from the group consisting of radiations, Ultra violet rays, gamma rays, radioactivity, heat shock and cold shock conditions in the environment.

In another embodiment, the bioactivity of growth promoter compounds is detected by profuse root initiation, root thickening, callusing and regeneration during a period ranging between 7 to 15 days.

In yet another embodiment, the bio-activity of growth inhibiting compounds is detected by absence of root initiation, wilting, withering within 2 weeks.

In yet another embodiment, the bioactivity of weedicidal compounds is detected by necrosis and death of the explant within a period of 3 days.

Further, in another embodiment, the bioactivity of antimitotic and anticancerous compounds is detected by reduction in cell division and miotic index within 7 days.

Further, in yet another embodiment, the bioactivity of cytotoxic compounds is detected by complete loss of chlorophyll within 3 days.

In another embodiment, the response to abiotic stress factors is detected by lethality after treating for 2 hours or more with the UV radiation within a period of 15 days.

In yet another embodiment, the response to abiotic stress factors is detected by absence of shoot proliferation and reduction in elongation and rooting, and increase in lethality at a dose greater than 5 Kr gamma radiation within a period of 15 days.

In yet another embodiment, the response to abiotic stress factors is detected by temperature sensitivity of Bacopa monnieri plants at a temperature of 80°C. for 20 seconds or more within a period of 7 days.

In yet another embodiment, response to abiotic stress factors is detected by temperature sensitivity of Bacopa monnieri plants at a temperature of 50° C. for 3 min or more within a period of 7 days.

In another embodiment, the response to abiotic stress factors is detected by temperature sensitivity of Bacopa monnieri plants at a temperature of −80° C. for 4 min or more within a period of 7 days.

In yet another embodiment, the response to abiotic stress factors is detected by temperature sensitivity of Bacopa monnieri plants at a temperature of −20° C. for 10 min or more within a period of 7 days.

In the other embodiment, the method tests abiotic stress factors and bioactivity of the natural, synthetic, semisynthetic compounds using Bacopa monnieri plant substantially as here in described and illustrated with reference to examples and photographs.

### BRIEF DESCRIPTION OF THE ACCOMPANYING DRAWINGS

1. Photograph 1 shows inoculated tubes in a half-transparent box.

2. Photograph 2 shows aerial root formation in case of cuttings upon auxin treatment.

3. Photograph 3 shows inhibition of root initiation and effect on aerial parts in terms of wilting of leaves.

4. Photograph 4 shows loss of chlorophyll in lower leaves of the twig

### DESCRIPTION OF THE INVENTION

In order to use Bacopa plant/plant parts for root initiation and shoot elongation, it is mandatory that the conditions should be standardized for efficient root initiation and elongation. The basal medium for studying the response was MS 0 (Murashige T and Skoog F, 1962, Physiol. Planta. 15: 473-497) supplemented with test compounds along with a control of just the basal medium. Measured 0.5 ml medium was poured into 1.5 ml graduated microcentrifuge tubes. Into this, twig cutting of 2.5 cm were inoculated in 20 replicates for each type of medium. These inoculated tubes were put into a half transparent (top lid) box allowing air passage through sterile cotton plugs such that the medium containing portion of the tubes where roots would be initiating is inserted into the stand holes made from a thermocol sheet (Refer Photograph 1 of Sheet #1 ). This set up was helpful in preventing the light exposure to the root portion while at the same time aerial parts got sufficient light and aeration. Such boxes were incubated at normal ambient temperature of 25-28° C. with 14 hours light and 10 hours dark cycle. The root initiation and shoot elongation was recorded from day 2 to 14 every 24 hours. Efficient root initiation was observed on MS basal medium only or that supplemented with ¼th nitrogen level in addition to normal concentrations. The earliest response was seen was on day 3 in case of medium containing ¼th level of nitrogen followed by medium containing normal level of nitrogen, while in case of zero nitrogen, initiation was observed only after 8 days. Thus, the medium with ¼ nitrogen level of standard MS basal medium was selected for root assays and was referred as MSR (rooting) medium.

In case of the treatment with the abiotic stress factors the plant parts/plantlets were taken into sterile petri plates or test tube and after the treatment again inoculated into MS basal medium and incubated at 25-28° C.

The present invention is particularly described with reference to the accompanying drawings and examples which are provided to illustrate the invention and should not be considered to restrict the scope of the invention.

### EXAMPLES

### Example 1

To find out the effect of growth promoters such as Indole 3 acetic acid (IAA), 2,4-Dichlorophenoxy acetic acid (2,4-D), and cytokinins such as 6-Benzyl Amino Purine (6-BAP) and Kinetin, the leaf, internode and twig cuttings were inoculated into MS 0 medium containing 1 mg/ml of the above said compounds and the responses were recorded up to two weeks in terms of rooting, callusing and shoot regeneration (Table 1). This defined the typical response, which was obtained in individual examples/treatments. The unique features was aerial root formation in case of cuttings upon auxin treatment as shown in (Photograph #2 of sheet #1 ). On the other hand, in case of cytokinin BAP green callus has formed, followed by shoot regeneration from leaf and internodal explant. The findings were suggestive of the use of these responses in assessing compounds for cytokinin and auxin like activities.

Table 1 summarizes the response recorded up to two weeks in terms of rooting, callusing a shoot regeneration.

TABLE 1

| | Response observed | | |
|---|---|---|---|
| Treatment | Leaf | Internode | Cutting |
| MS 0 and | Root Initiation | Root Initiation | Aerial Root |
| 2,4-D | Root Thickening | Root Thickening | Formation |
| | Callus Initiation | Explant Swelling | |
| MS 0 and | Root Initiation | Root Initiation | Aerial Root |
| IAA | (Profuse) | (Profuse) | Formation |
| MS 0 and | Callus Initiation | Callus Initiation | Root Initiation |
| BAP | Shoot | Shoot | Root Thickening |
| | Regeneration | Regeneration | |
| MS 0 and | Root Initiation | No Response | Root Initiation |
| Kinetin | | | Root Elongation |
| MS 0 only | Root Initiation | No response | Root Initiation |
| | Root Elongation | up to 15 days | |

### Example 2

The effects of growth inhibitors were also tested using the protocol as described in Example 1. In this experiment the twigs were in the basal MS media containing Nalidixic acid (Nal) at the rate of 10 μg/ml and 100 μg/ml and the effects were observed after 10 days of inoculation. In case of the 10 μg/ml concentration of nalidixic acid roots got initiated from the submerged nodes but their growth was less than the control MS basal medium. While in case of 100 μg/ml concentration of nalidixic acid very small roots arose from submerged node and their growth was completely arrested.

### Example 3

The conditions were standardized for root tip assays to determine the effect of compounds like taxol on cell division. The following example gives the experimental procedure, which was followed and found suitable for determining cell division initiation in the roots initiated from twig cuttings of Bacopa as described in general methodology on day 7. The fresh initiated roots in MSR medium were dipped in micro-centrifuge tubes containing MS broth with solvent (DMSO or likewise), the compound (taxol dissolved in DMSO or likewise) and control (MSR broth). The dipping treatment was continued for 3 hours after which the root tips were cut and fixed in a 1:3 solution of glacial acetic acid and absolute alcohol and the fixation was continued for 24 hrs at room temperature. For long term storage such roots could be stored in 90% ethanol. For further processing these fixed roots tips were treated with 5 N HCl for 10 min followed by thorough washing with sterile distilled water (3 to 4 times). This followed the treatment with iron allum (1%) for 30 min and then staining with matured haemotoxylin for 2.5 hrs. The mounting was done in 45% acetic acid by gently tapping and the slide was observed under the microscope (400 to 1000 times magnification). Mitotic index was estimated based on the number of dividing cells in 10 randomly selected fields. Taxol for instance, was found to significantly reduce the mitotic index (Table 2) indicating the applicability of the procedure in detecting cell division inhibitors

TABLE 2

| | | Total No. | | | | | |
|---|---|---|---|---|---|---|---|
| | | of | | | | | |
| | | cells | Number of cells in phase Mitotic* | | | | |
| Compounds/ | | observed | Pro | Meta | Ana | Telo | Index |
| 1. | Control | 65 | 03 | 06 | 01 | 04 | 18.4 + |
| | | | | | | | 4.95 |
| 2. | Taxol(10 μg/ml) | 70 | 01 | 03 | 01 | 00 | 7.14 + |
| | | | | | | | 1.52 |
| 3. | Trichothecene- | 37 | 03 | 00 | 00 | 00 | 8.1 + |
| | T14-(10 μg/ml) | | | | | | 1.45 |

*Taxol and T-14 at 100 μg/ml inhibited root initiation itself hence no mitotic index was determined.

### Example 4

Simultaneously, like for the root tip assays, the procedure was also developed to determine the effect on root initiation itself by inhibitory compounds like taxol using twig cuttings. In this example, instead of pre-initiated roots, the fresh twig cuttings were directly subjected to the treatment of compound included in the rooting medium. For example, taxol was added to MSR at the rate of 1,10 and 100 μg/ml in the 0.5 ml agar medium prepared in microcentrifuge tube as described otherwise in Example 1. The treatments included compounds (taxol and likewise), solvent (DMSO and likewise) and control (plain MSR). The twig cuttings were surface sterilized and inoculated into the toxic and control media. These were then incubated as in Example 1 for root initiation and growth up to day 15. Taxol at the concentration of 10 and 100 μg/ml inhibited the complete root initiation and also had a visible effect on the aerial parts in terms of wilting of the leaves (Refer Photograph #3 of Sheet #2). The findings of this experiment further substantiated the usability of Bacopa test procedure not only for cytological parameters but also for visible phenotypic expression, for instance, root initiation and shoot health as manifested here in terms of wilting.

### Example 5

To test cytotoxic effects of mint oil and other essential oils, the twigs were inoculated into the MS basal medium. Here, the mint oil was either added into the medium at the rate of 1 μl/10 ml or sprayed on the in-vitro plants at the rate of 250 μl/ml. In case where mint oil was added into the medium i) no root initiation was observed from the twigs, ii) wilting of twigs occurred after two days of inoculation, and iii) chlorosis occurred after 5 days. While in case where mint oil was sprayed, i) wilting was observed on next day and ii) chlorosis recorded after 3 days of inoculation.

### Example 6

In order to check the effect of radiations (Ultra Violet and gamma-Rays) on the different parts of the Bacopa plant, plantlets were exposed to Ultra Violet (UV) radiation with a 312 mn germicidal lamp from a 30 cm distance in a dark chamber for different time periods ranging from 0 hour to 12 hour and readings were recorded upto 15 days. Maximum lethality was observed in case of total plant with overnight exposure while, in case of the exposed plantlets with exposures of 1, 2, 4 and 8 hours, reversible lethality was observed (Table 3). It means the original plant, which was exposed to UV radiation, died but the buds were alive and later proliferated into new shoots under normal conditions. Similarly, the plantlets were exposed to gamma-radiations at 0 Kr to 100 Kr. Lethality (browning of leaf and stem tissue) was observed 15 days after exposure at the doses of 5 Kr and above The detail data is given in the Table 4.

TABLE 3
Effect of Ultra Violet radiation on
in-vitro raised Bacopa monnieri plantlets

| Dose | Lethality |
|---|---|
| 0 hour | − |
| 1 hour | − |
| 2 hours | + |
| 4 hours | + |
| 5 hours | + |
| 8 hours | + |
| 10 hours | + |
| overnight | + |

+ = Occurred;
− = Did not occur

TABLE 4
Effect of γ-rays on in-vitro raised Bacopa monnieri plantlets.

| | Shoot | Shoot | | |
|---|---|---|---|---|
| Dose | Proliferation | Elongation | Rooting | Lethality |
| 0 Kr | + | 5 cm | 100% | − |
| 1 Kr | + | 5 cm | 100% | − |
| 2 Kr | − | 3 cm | 50% | − |
| 5 Kr | − | 2.5 cm | 10% | + |
| 10 Kr | − | 2 cm | − | + |
| 20 Kr | − | 1.5 cm | − | + |
| 30 Kr | − | 1.5 cm | − | + |
| 50 Kr | − | 1.5 cm | − | + |
| 100 Kr | − | 1.5 cm | − | + |

+ = Occurred
− = Did not occur

### Example 7

Bacopa monnieri plant system was also used for carrying out temperature sensitivity assays by subjecting the plantlets to heat and cold shocks. Heat shocks were given at 50° C. and 80° C. and cold shock at −20 and −80° C. for varying time period ranging from 10 seconds to 30 minutes. Heat shock at 50° C. with the exposure for 3 minutes and more killed all the plants while at 80° C. plants died after the exposure for just 20 seconds. Plants demonstrated sensitivity to cold shock of −20° C. and −80° C. within 10 minutes and 4 minutes respectively (Table 5).

TABLE 5
Temperature sensitivity of Bacopa monnieri plant

| | Time Period | | | | | | | | | | |
|---|---|---|---|---|---|---|---|---|---|---|---|
| Temperature | 10″ | 20″ | 30″ | 1′ | 2′ | 3′ | 4′ | 5′ | 10′ | 20′ | 30′ |
| −20° C. | − | − | − | − | − | − | − | − | + | + | + |
| −80° C. | − | − | − | − | − | − | + | + | + | + | + |
| 50° C. | − | − | − | − | − | + | + | + | + | + | + |
| 80° C. | − | + | + | + | + | + | + | + | + | + | + |

″ = Seconds; ′ = Minutes
+ = Death of plants; − = Plants are alive

### Example 8

We also planned the experiments to determine if this procedure can depict a clear cut manifestation of cytotoxic/weedicidal effect of compounds. For this purpose we used a known essential oil of Mentha arvensis which is rich in toxic but useful component menthol as the treatment. The procedure involved was same as in above examples with the exception that menthol was added to the medium and oil was sprayed on these twigs inoculated into the medium. The treatment within 3 days resulted in to complete loss of chlorophyll in the lower leaves which further within a week time covered whole twig (Refer Photograph #4 of Sheet #2). The procedure therefore, showed its use also in assessing and screening the phytotoxic chemicals.

### Example 9

The above examples clearly established the use of Bacopa test procedure in detecting compounds with various biological activities such as root initiation inhibition, cell division inhibition (or anticancerous), growth promotion (shoot as well as root proliferation), growth inhibition (wilting, root stunting etc.), in vitro cell/ tissue differentiation (callusing, shoot and root regeneration) and phytoxicity (Table 6). These findings were strongly supportive of accepting our test system as a unique one which by a single basic procedure can detect so many activities ranging from cytological mitotic effects to phenotypically visible plant growth parameters. We therefore, used this example to apply on various unknown and uncharacterized plant compounds and extracts just with the objective to assess the applicability of our test in a large screening programme. The visible effects of these compounds established the usability of the procedure in detecting and differentiating bioactivities of compounds.

TABLE 6
Effects of some unknown compounds on Bacopa monnieri plant

| | Effect/Response | | | | |
|---|---|---|---|---|---|
| | | | | | Expected |
| Unknown | | | | Chloro- | activity in |
| test | Shoot | Root | | phyl | test |
| compound | proliferation | elongation | Lethality | 1 loss | compound |
| A01 | − | + | − | − | Growth |
| | | | | | promoter |
| F01 | + | + | − | − | Growth |
| | | | | | promoter |
| 113 | − | − | + | − | Growth |
| | | | | | inhibitor |
| MRP | − | − | − | + | Cytotoxic, |
| | | | | | Weedicidal |
| Art 400 | − | − | + | + | Cytotoxic, |
| | | | | | Weedicidal |

### Advantages of the Invention

The applicants used the fast propagating strain of the plant Bacopa monnieri for the first time to assess effect of any compound or physical factor on the responses of the plant and plant part. The system is unique because:

1. Any aerial part (internode, node, buds and leaves) can be used to initiate roots in the medium/buffer.

2. The root initiation is possible throughout the year irrespective of the seasons by just maintaining the temperature.

3. The initiated roots can be tested on the medium containing chemical compounds in volumes as small as 0.5 ml in microcentrifuge tubes permitting thousands of samples being tested in a single experiment.

4. The response of Bacopa plant or plant parts particularly leaves, internodes and twigs can be clearly differentiated for the auxin-like Vs. Cytokinin-like activities.

5. Testing of anticancerous agents affecting the mitotic index as well as colchioine type effects can be visualized in the Bacopa test system.

6. Weedicidal/cytotoxic effect can also be detected in the Bacopa test system.

7. The response of Bacopa twigs can be used directly to assess the presence of physical factors in the environment including radioactive substances, radiations like UV/gamma-rays and sudden heat or cold shocks.

8. The usual root tip assay can be done with Bacopa in a much simpler and rapid way by initiating and monitoring the roots on the lower end of aerial twings within 8-15 days time.

Thus the present invention provides new assay procedure which can be used to detect wide spectrum of activities and parameters in a single test system. Our invention is also much more sensitive, fast and can test the effect of compounds of different activities in the same system, in contrast to other available individual tests. This procedure of the invention thus, can replace number of separate tests required for different compounds and activities.

1. A method for testing abiotic stress factors and bioactivity of natural, synthetic and semisynthetic compounds using Bacopa monnieri plant, which comprises growing the said plant and plant parts aseptically in MS 0 basal medium with agar in microcentrifuge tubes by adding the compounds to be tested either to the culture media or by spraying the said compounds on the plant or plant parts or on the said medium to detect the distinct morphological and cytological responses.

2. A method as claimed in claim 1, wherein the said compounds used are either in pure or crude extract form.

3. A method as claimed in claim 1, wherein the group of morphological responses to be detected may be selected from callus initiation, shoot induction, root induction, necrosis and death.

4. A method as claimed in claim 1, wherein the cytological response to be detected may be selected from the group comprising mitotic index and C-mitosis.

5. A method as claimed in claim 1 wherein the compound used for bio-te sting includes growth promoters selected from the group comprising auxins and cytokinins.

6. A method as claimed in claim 1 wherein the auxins used are selected from the group comprising but not limited to Indole 3 acetic acid and 2, 4-Dichlorophenoxy acetic acid.

7. A method as claimed in claim 1, wherein the cytokinins used are selected from the group comprising but not limited to 6-Benzyl amino purine and kinetin.

8. A method as claimed in claim 1, wherein the compounds used for biotesting includes growth inhibitors selected from but not limited to the group comprising nalidixic acid and abscisic acid.

9. A method as claimed in claim 1, wherein the compounds used for biotesting includes weedicidal compounds.

10. A method as claimed in claim 1, wherein the weedicidal compound includes 2,4-Dichlorophenoxy acetic acid.

11. A method as claimed in claim 1, wherein the compounds used for biotesting comprise antimitotic and anticancerous compounds.

12. A method as claimed in claim 11, wherein the antimitotic and anticancerous compounds are selected from the group comprising but not limited to taxol, trichothecene, vincristine, vinblastine and nalidixic acid.

13. A method as claimed in claim 1, wherein the compounds used for biotesting include cytotoxic compounds.

14. A method as claimed in claim 1, wherein the cytotoxic compounds are selected from the group consisting of but not limited to menthol, mint oil, artemisia oil, basil oil and essential oil components.

15. A method as claimed in claim 1, wherein the abiotic stress factors used are selected from the group consisting of radiation, Ultra violet rays, gamma rays, radioactivity, heat shock and cold shock conditions in the environment.

16. A method as claimed in claim 1 wherein bioactivity of growth promoter compounds is detected by profuse root initiation, root thickening, callusing and regeneration formed during a period ranging between 7 to 15 days.

17. A method as claimed in claim 1, wherein bio-activity of growth inhibiting compounds is detected by absence of root initiation, wilting, withering within 2 weeks.

18. A method as claimed in claim 1, wherein bioactivity of weedicidal compounds is detected by necrosis and death of the explant within a period of 3 days.

19. A method as claimed in claim 1, wherein bioactivity of antimitotic and anticancerous compounds is detected by reduction in cell division and miotic index within 7 days.

20. A method as claimed in claim 1, wherein bioactivity of cytotoxic compounds is detected by complete loss of chlorophyll within 3 days.

21. A method as claimed in claim 1, wherein the response to abiotic stress factors is detected by lethality after treating for 2 hours or more with the UV radiation within a period of 15 days.

22. A method as claimed in claim 1, wherein the response to abiotic stress factors is detected by absence of shoot proliferation and reduction in shoot elongation and rooting, and increase in lethality at a dose greater than 5Kr gamma radiation within a period of 15 days.

23. A method as claimed in claim 1, wherein the response to abiotic stress factors is detected by temperature sensitivity of Bacopa monnieri plants at a temperature of 80° C. for 20 seconds or more within a period of 7 days.

24. A method as claimed in claim 1, wherein the response to abiotic stress factors is detected by temperature sensitivity of Bacopa monnieri plants at a temperature of 50° C. for 3 min. or more within a period of 7 days.

25. A method as claimed in claim 1, wherein the response to abiotic stress factors is detected by temperature sensitivity of Bacopa monnieri plants at a temperature of −80° C. for 4 min or more with a period of 7 days.

26. A method as claimed in claim 1, wherein the response to abiotic stress factors is detected by temperature sensitivity of Bacopa monnieri plants at a temperature of −20° C. for 10 min or more within a period of 7 days.
